(19) United States
(12) Patent Application Publication
(10) Pub. No.: US 2024/0024085 A1
(43) Pub. Date: Jan. 25, 2024

(54) DEVICE FOR INJECTING FLUID INTO A BIRD

(71) Applicant: Desvac, Saint-Barthelemy D'Anjou (FR)

(72) Inventor: Philippe Magniaux, Libourne (FR)

(21) Appl. No.: 18/028,412

(22) PCT Filed: Sep. 14, 2021
(86) PCT No.: PCT/FR2021/051570

§ 371 Date: Mar. 24, 2023

(30) Foreign Application Priority Data
Sep. 25, 2020 (FR) 20306092.6

(51) Int. Cl. A61D 1/02

(52) U.S. Cl. CPC A61D 1/025

(57) ABSTRACT

The present invention relates to a device for injecting fluid into a bird, the device having a main body (11), which comprises an injection assembly comprising an injection needle and at least one syringe, the needle being movable between a retracted position and an injecting position located in an injection zone positioned outside the main body (11). According to the invention, said device comprises:—a guide element (12) positioned on an exterior surface of the main body (11), the guide element (12) comprising a base (13) and side edges (14) extending from said base, at least the upper part of the back of the bird to be vaccinated being intended to rest against said base (13) and to be moved, head first, along a guide axis (15) defined by the guide element (12), to the injecting position,—the guide element (12) being additionally configured to keep at least the neck of the bird aligned or substantially aligned along said guide axis (15) in the injection zone—the injection needle being arranged such that when it is moved between the retracted and injecting positions, it moves along an axis of movement parallel to the exterior surface of the base, or at an angle relative to said exterior surface, said axis of movement extending, away from the main body, beyond an exclusion zone surrounding the guide axis (15).

### TECHNICAL FIELD

The present invention relates to a device for automatically injecting a fluid substance, such as a vaccine, into a bird, in particular a one-day-old chick.

### PRIOR ART

In order to face a constantly-growing demand for food, animals originating from intensive farming systems have grown in number.

Good health in these animals, in particular to guarantee suitable yields, involves preventing the appearance of health problems, such as the emergence of diseases within the farm. This prevention can be carried out, in particular, by vaccinating the animals.

It is thus known in the breeding of poultry, to inoculate chicks with a vaccine when they are one day old, or between one (1) and twenty-one (21) days old.

The handling of these chicks is easy, as they are particularly docile, and the fineness of their down guarantees easy introduction of the injection needle into the skin of the chick and consequently the reliability of the injection.

To deliver injections in as short a time as possible, automatic vaccine injection apparatuses are typically implemented.

By way of example, patent application WO 2018/167562 A1 in the name of the present applicant discloses an automatic vaccination apparatus.

This apparatus comprises in particular a frame having a bent front panel delimiting an injection zone, an injection assembly being housed in this frame, this injection assembly comprising one or more syringes and an injection needle, and an actuation button placed in the angle formed by the bent front panel.

When the chick's body is pressed against this actuation button with sufficient force, a drive mechanism moves the needle, projecting the needle out from the frame to ensure the administration of an injection into the body of the chick.

Although it gives good results, this apparatus can also be improved.

First, each chick is presented in lying-down position in the injection zone of the apparatus, for the purpose of injecting the vaccine.

However, this unnatural position for a chick is not only a source of stress to it, but it also requires the performance of repetitive movements of the forearm and corresponding wrist of the operator to ensure that the chick is properly oriented.

Thus, the appearance of musculoskeletal disorders (MSDs) in that operator can be observed over time.

Such an apparatus also allows the chick to be presented on only one single side of the apparatus.

However, such a geometry of the apparatus necessarily constitutes a limit on the number of chicks that can be treated within a given time.

Aside from increased fatigue that the operator can feel due to the repetitive gestures performed to treat the chicks, vaccination is made more complex for an operator, when the side of the apparatus allowing the insertion of a chick does not correspond to the handedness (right or left) of that operator.

Furthermore, it has been observed that the positioning of the chick in the injection zone of the apparatus is free, which given the injection rates attained, entailed a risk not only to the animal's health, but also of accidental injury to the operator.

Losses related to injection errors are of course the cause of an increase in costs, which is not desirable for the operator.

There is therefore a pressing requirement for a device for injecting a fluid substance into an animal to be injected, the original design of which makes it possible to overcome the disadvantages of the prior art described above.

### SUBJECT MATTER OF THE INVENTION

The present invention aims to overcome the disadvantages of the prior art by proposing a device for automatically injecting a liquid into a bird, simple in its design and in its operating mode, allowing a precise and secured injection of that liquid.

Another object of the present invention is such an injection device, allowing faster rates of vaccination.

Yet another object of the present invention is such an injection device offering increased protection to the operator, while respecting the well-being of the birds.

### DISCLOSURE OF THE INVENTION

To that end, the present invention relates to a device for injecting fluid into a bird, that device having a main body, which comprises an injection assembly comprising an injection needle and at least one syringe, the needle being movable between a retracted position and an injecting position located in an injection zone positioned outside that main body.

According to the invention, said device comprises:
a guide element placed on an exterior surface of the main body, said guide element comprising a base and side edges extending from this base, at least the upper part of the back of the bird to be vaccinated being intended to rest against said base, and to be moved, headfirst, along a guide axis defined by the guide element, to the injecting position,
said guide element being additionally configured to keep at least the neck of the bird aligned or substantially aligned along said guide axis in the injection zone,
said injection needle being arranged such that, during its movement between said retracted and injecting positions, it moves along an axis of movement parallel to the exterior surface of the base, or at an angle relative to said exterior surface, said axis of movement extending, away from the main body, beyond an exclusion zone surrounding the guide axis.

Advantageously, the part of the body of the bird wherein the injection must be administered, being held in position by the guide element at the injection zone, a greater precision is obtained in the injection carried out and a lower risk to the health of the bird.

Such a holding in position also makes it possible to define a protective space, referred to as the exclusion zone, wherein the injection needle is absent when it is partially outside the main body, or wherein this needle does not penetrate during its movement between the retracted and injection positions, this space being intended to house sensitive parts of the body of the bird, in particular the vertebral column thereof.

This protective space is not necessarily symmetrical about the guide axis, as the person skilled in the art understands that this protective space is necessary in the zone where the injection needle moves out from the main body.

By way of example, the injection zone is defined as being the space extending at least around the path traveled by the injection needle when it penetrates into the upper end of the body of the living animal to the injection point.

Purely by way of illustration, this guide axis is an axis of symmetry of the guide element.

The term “bird” is understood here to mean any avian species, such as birds from the class of Ayes, that is vertebrates which are feathered, winged, bipedal, endothermic (warm-blooded) and can lay eggs. In the context of the present invention, the term more particularly refers to birds of economic and/or agronomic interest, such as poultry (for example, chickens, turkeys, hens, guinea fowl, quails, partridge, and pigeons), migratory fowl (for example, ducks and geese) and ornamental birds (for example, swans, parrots, and parakeets).

Such an injection device finds applications in particular in the field of vaccinating chicks within a few days following their birth, that is, between one (1) and fourteen (14) days after hatching. Preferentially, vaccination is carried out on birds of between one (1) and seven (7) days of age after hatching. More preferentially, vaccination is carried out on birds of between one (1) and two (2) days of age after hatching, that is, 48 hours after hatching. Even more preferentially, vaccination is carried out on birds at one (1) day of age, that is, 24 hours after hatching.

Advantageously, such a configuration of the injection device allows free access, or even entirely open access, to the guide element such that the operator can use both hands to put the birds into the injection position.

The vaccination rate allowed by this automatic injection device is thus significantly improved.

This guiding element can be affixed or secured to the outer surface of the main body, this outer surface also being bent to impose a particular tilt to the head of the bird in order to release access to its neck with a view to inserting the injection needle.

The injection is preferably carried out in the bottom of the neck or the top of the back of the animal to be injected, preferentially just below the neck.

Alternatively, this outer surface can itself be shaped to define a recessed guiding element in the main body, this guiding element having a base against which the part of the body of the bird wherein the injection must be administered is pressed.

According to one embodiment of this injection device, the part of the base of the guide element placed at the path along which the needle moves between the retracted and injection positions is flat or substantially planar.

According to another embodiment of this injection device, the axis of movement of said needle between said retracted and injecting positions is inclined by an angle of between [0, 4°] relative to the exterior surface of said base.

This ensures that the injection needle cannot penetrate deep into the animal's body during the administration of the injection. By way of example, this angle of inclination between the axis of movement of the needle and the base is 2+/−2°. Such an angle can be obtained in various ways, for example by keeping the base fixed and pivoting the injection needle or even keeping the orientation of the injection needle unchanged, and pivoting the base. Of course, it is also possible to pivot the needle and the base to obtain such an angle of inclination of the axis of movement of the needle relative to the base of the guide element.

According to yet another embodiment of this injection device, this exclusion zone is a space extending over 2 to 4 mm around said guide axis.

In other words, in the injection position, the free end of the injection needle is placed at a distance of between 2 and 4 mm from the guide axis.

Purely by way of illustration, the guide element being shaped to align, or substantially align, at least the cervical spine of the animal with this guide axis, for example, by a constriction of this guide element holding its neck in position, the needle is arranged such that the path it travels between the retracted and injection positions is laterally offset by a distance d relative to this guide axis, and consequently, the position of the cervical spine of that bird.

It is thus advantageously ensured that the injection needle is only introduced into soft neck tissues or from the top of the bird's back and that no contact with the animal's vertebral column is carried out during injection.

More generally speaking, this guide element is configured such that the spine of the bird is placed in the exclusion zone, when at least the upper part of the back of the bird to be vaccinated is pressed against the base and at least the neck of that animal is kept aligned or substantially aligned along this guide axis in said injection zone.

According to yet another embodiment of this injection device, the axis of movement of said injection needle being contained in a plane parallel to the base of the guide element, this axis of movement and the guide axis are collinear or this axis of movement of the needle diverges from this guide axis.

This axis of movement of the needle can thus be parallel to the guide axis by being spaced apart, or offset, from the latter so as not to pass through or be within the exclusion zone.

Alternatively, this axis of movement of the injection needle can be placed at an angle relative to the guide axis but moving away, or even diverging, from the latter, going towards the exterior of the main body such that the needle is indeed outside the exclusion zone when it is located in the injection zone.

According to yet another embodiment of this injection device, this device is configured such that its main body being placed on a flat horizontal surface, the bird is injected in the vertical, or substantially vertical, position, that is, in the upright position.

The head of the bird being directed toward the upper part of the main body, that is, the bird being moved in the guiding element head the first, the weight of the body of this bird ensures that it is positioned vertically and that the bird is presented at the injection point in the upright position.

The outlet of the injection needle can then be done vertically from top to bottom.

It is also observed with such an embodiment that the risks for the operator to develop chronic disease related to the handling of chicks are considerably limited

According to yet another embodiment of this injection device, in line with the injecting position, the distance separating the free end of said injection needle from the exterior surface of the base determines subcutaneous injection into the neck of said bird.

Preferably, this distance is typically between three (3) and seven (7) mm.

This ensures that the needle cannot penetrate deep into the animal's body during the administration of the injection.

Tests carried out on one-day-old chicks demonstrated the precision of the injection carried out with the device of the invention, in particular the absence of blood when removing the injection needle (“bloody neck”)

Purely by way of illustration, the movement of the needle between the retracted and injection positions is on the order of 23 to 27 mm.

According to yet another embodiment of this injection device, a part of the body of this guide element is configured to form a protective barrier placed at least on either side of the path along which said needle moves between the retracted and injection positions.

Advantageously, the edges of the guide element are thus configured to define such a protective barrier, these edges also forming lateral guide rails.

The safety of the operator is thus substantially reinforced. Any risk of accidental pricking of the operator is eliminated because of the elevation of the proposed protective barrier.

According to yet another embodiment of this injection device, this guide element comprises a first part of the body with a flared shape and a second part of the body forming a constriction, the first part of the body being arranged to provide guidance of the upper part of the bird's body into which the injection must be administered, towards this constriction.

This constriction is advantageously positioned at the injection zone.

The first body part of this guiding element is therefore intended to guide, preferably gradually, the upper part of the body of the bird to the second part of the body of the guiding element forming a constriction. Once received in this constriction, the upper part of the bird's body, in particular its neck, is then wedged in order to receive the injection needle.

Such guidance of the top of the body of the animal to be injected also allows for blind vaccination of the birds.

By way of example, the operator can be placed behind the injection device or in any other position around this apparatus, that is, over the 360° around the latter.

Advantageously, the body part of the guide element forming a constriction is configured to be adjustable in particular in its transverse dimension, or else in width, to adapt to the size of the bird.

According to yet another embodiment of this injection device, the main body comprises recesses arranged laterally to this guide element in order to facilitate the insertion of the bird to be injected by the operator into this guide element.

According to yet another embodiment of this injection device, this main body comprises at least one force sensor, preferentially two force sensors.

The movement of the needle is controlled only by the triggering of this sensor, or of these sensors simultaneously.

Preferably, these force sensors are advantageously arranged relative to the guiding element in order to ensure an alignment, or substantially an alignment, of the part of the vertebral column of the bird located at least in this injection position with the outer surface of this base, when said bird is pressed against the guiding element and bearing against said force sensors.

According to yet another embodiment of this injection device, the lower end of the guide element is in communication with a chute ensuring the removal of the bird after the injection has been administered.

Once the injection has been administered, the operator can simply release the bird, which can fall into a storage basket or be recovered by the chute to be discharged to a storage area or another work area.

Preferably, this chute is orientable up to 180°. This free orientation of the chute can be achieved, for example, about an axis of rotation parallel to the guide axis defined by the guide element.

According to yet another embodiment of this injection device, each injection assembly also comprises a linear actuator, this linear actuator is a hydraulic, pneumatic or electric actuator.

According to yet another embodiment of this injection device, the needle is offset relative to the syringe or to the syringes.

The assembly formed by a manifold and the needle support is thus movable and independent of the unit consisting of the cylinder and the damper.

This assembly can easily be dismantled for maintenance or changing an injection needle.

Since each injection needle is thus not placed in the extension of its syringe, a more compact injection device is therefore obtained.

According to yet another embodiment of this injection device, this device comprises two syringes and an injection needle.

Advantageously, the actuation of a control button placed on the main body of the injection device makes it possible to switch from a single to a double injection (activation of one or both cylinders).

According to yet another embodiment of this injection device, the syringe(s) are arranged in the main body so as to ensure the discharge of air bubbles which could form in the corresponding syringe.

According to yet another embodiment of this injection device, the angles of the constituent parts of this injection device with which the bird and/or the operator are likely to come into contact are rounded.

This prevents accidentally injuring the animal to be injected, as well as the operator.

Furthermore, cleaning the injection device is easier.

According to yet another embodiment of this injection device, the upper end of said main body has an elbow to impose a particular inclination on the head of the bird, this elbow comprising one or more holding elements configured to keep the head of the bird to be vaccinated in position.

It is thus ensured that the head of the bird is wedged in a position from which it can no longer can turn its head. It is thus the entire upper part of the body of the living animal to be vaccinated which is kept aligned or substantially aligned along the guide axis.

### BRIEF DESCRIPTION OF THE DRAWINGS

Other advantages, aims and particular features of the present invention will become apparent from the following description, made, for explanatory purposes and in no way limiting, with reference to the appended drawings, in which:

FIG. 1 is a profile view of an automatic injection device for a liquid substance in a bird according to a particular embodiment of the present invention;

FIG. 2 is a partial perspective view of the injection device of FIG. 1 showing the arrangement of the upper end of the guide element in relation to the actuation buttons;

FIG. 3 is a partial perspective view of the injection device of FIG. 1 showing the lower part thereof;

FIG. 4 is a partial front view of the injection device of FIG. 1 showing the arrangement of the upper end of the guide element in relation to the actuation buttons;

FIG. 5 is a longitudinal sectional view of the injection device of FIG. 1;

### DESCRIPTION OF AN EMBODIMENT

The drawings and the following description essentially contain elements of a certain nature. They may therefore not only serve to better understand the present invention, but also contribute to its definition, where appropriate.

First, it should be noted that the figures are not to scale.

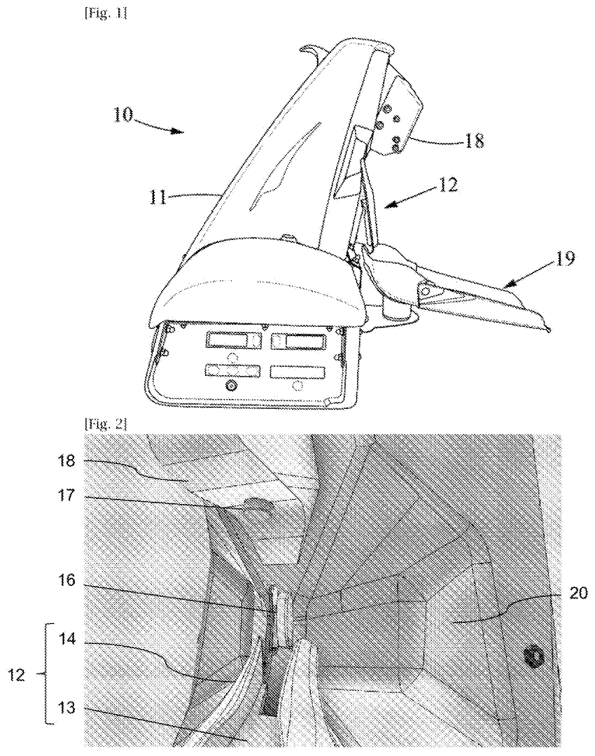

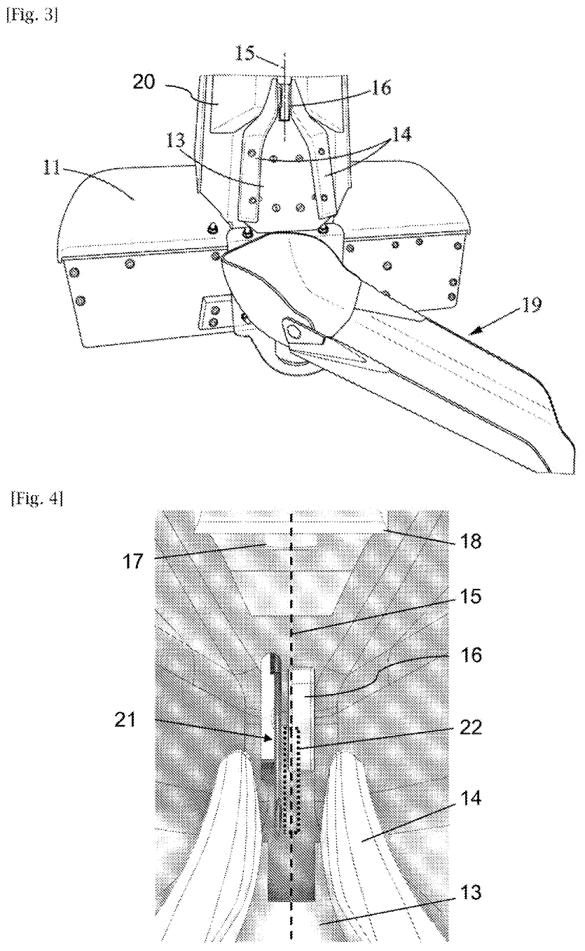

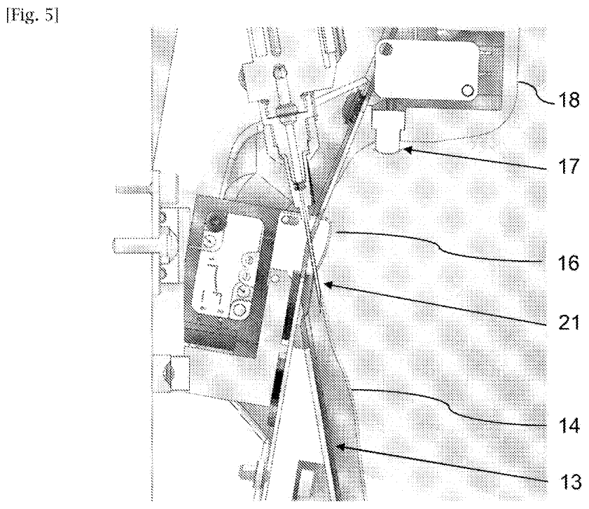

FIGS. 1 to 5 schematically show a device for injecting a liquid substance into a bird according to a particular embodiment of the present invention.

This injection device 10 comprises a frame 11 inside which an injection assembly is placed. This injection assembly comprises, in a known manner, one or two syringes and an injection needle.

Each syringe is supplied with liquid substance by a reservoir (not shown), such as a flexible pouch, placed outside the frame 11. This reservoir which determines a storage volume wherein this liquid substance is received is supported by a pouch-holder (not shown), optionally a telescopic one. Here, it is connected to the inner volume of each syringe by a flexible conduit such as a flexible hose.

Each syringe is preferably oriented in the vertical, or substantially vertical position, discharging of the liquid substance upward, in order to allow the discharging of any air bubbles that would be formed.

The outer surface of this frame 11 comprises a support 12 for receiving the bird.

The body of the frame 11 has, on each side of this support 12, recesses, or even clearances, 20 to facilitate the movement of the operators thumbs without the operator being forced to bend his wrists. The insertion of a chick into the support 12 is thus made easier.

This support 12 comprises a base 13 and edges 14 extending from this base to the outside. These edges 14 laterally surround this base 13 by projecting therefrom.

This support 12 comprises a flared-shaped body portion in its lower part and a constriction-shaped body portion in its upper part, the flared-shaped portion opening into the constriction and making it possible to gradually guide the upper part (head and neck) of the bird towards this constriction.

The base 13 of the support 12 at this constriction has an elongated shape defining with the edges 14 a longitudinal axis 15.

The operator who has entered a bird for vaccination, therefore presses the upper part of the back of this animal against the base 13, the latter being surrounded by the edges 14.

The operator can then move that animal head-first along this longitudinal axis 15 to an injection position of the injection device.

The base 13 of the support 12 at the constriction also comprises a first actuation button 16, which is flat extending over at least a portion of this base. This actuation button 16 is activated when the neck of the bird, wherein the injection must be administered, is pressed against the base by being received in this constriction.

This constriction is configured to ensure a wedging into position of the neck of the bird so that its neck is aligned or substantially aligned along this longitudinal axis 15 at an injection zone defined by the movement of the injection needle 21 of the injection device from the main body 11.

A second actuation button 17 is placed on a bent part defining a stop 18 for the head of the bird.

When the upper part of the body of the bird is pressed against these two actuation buttons 16, 17 simultaneously with a sufficient force, a drive mechanism (not shown) placed in the frame 11 moves the injection needle 21, the latter passing from a retracted, or pulled-back, position inside the frame 11, to an injection position external to the frame 11.

In this injection position, the needle 21 is placed in the constriction of the support 12, such that an injection can be administered in the bottom of the neck of the bird. This neck is advantageously released and wedged in position when the neck of the bird is received in this constriction, the head of the bird is pressed against the bent part forming a stop 18 for the head of the bird.

Between these retracted and injection positions, the needle is movable in translation along an axis of movement parallel to the outer surface of the base of the support, while being parallel to the longitudinal axis 15.

Thus, the expulsion of the injection needle 21 happens vertically from top to bottom, being parallel to the outer surface of the base 13 of the support 12 such that the latter can in no way penetrate into the body of the bird.

In line with the injection position, the distance separating the free end of the injection needle 21 from the base 13 of the support 12 is 5+/−2 mm so that subcutaneous injection of the liquid substance to be administered into the neck of the bird is thus guaranteed.

Furthermore, the needle is eccentric relative to the longitudinal axis 15 of the support 12 so as not to inject the product into the vertebral column of that animal. An exclusion zone 22 is thus advantageously defined surrounding the longitudinal axis 15 and wherein the injection needle 21 cannot penetrate and consequently injure the animal.

The path followed by the injection needle during its movement between the retracted and injection positions is also enclosed by the edges 14 of the support 12. These edges 14 advantageously form a protective barrier for the operator to ward off accidental injury.

Advantageously, the base 13 of the support is slightly inclined to facilitate the insertion of the upper part of the body of the bird into the constriction.

The lower end of the support 12 is in communication with a chute 19 ensuring evacuation of the bird after the injection is administered.

The vaccinated bird can then be released by the operator, the chute having a curved shape damping its fall. The axis at which the bird falls and the axis of movement of the needle being aligned, the risk of injury of the bird is minimized. The bird lands on its feet, since it still remains in vertical or substantially vertical position.

1. A device for injecting fluid into a bird, the device having a main body (11), which comprises an injection assembly comprising an injection needle and at least one syringe, the needle being movable between a retracted position and an injecting position located in an injection zone positioned outside the main body (11), characterized in that it comprises:
a guide element (12) placed on an exterior surface of the main body (11), said guide element (12) comprising a base (13) and side edges (14) extending from this base, at least the upper part of the back of the bird to be vaccinated being intended to rest against said base (13), and to be moved, headfirst, along a guide axis (15) defined by the guide element (12), to the injecting position,
said guide element (12) being additionally configured to keep at least the neck of the bird aligned or substantially aligned along said guide axis (15) in the injection zone,
said injection needle being arranged such that, during its movement between said retracted and injecting positions, it moves along an axis of movement parallel to the exterior surface of the base, or at an angle relative to said exterior surface, said axis of movement extending, away from the main body, beyond an exclusion zone surrounding the guide axis (15).

2. The injection device according to claim 1, characterized in that the axis of movement of said needle between said retracted and injecting positions is inclined by an angle of between [0, 4°] relative to the exterior surface of said base (13).

3. The injection device according to claim 1, characterized in that said exclusion zone is a space extending over 2 to 4 mm around said guide axis (15).

4. The injection device according to claim 1, characterized in that the axis of movement of said injection needle being contained in a plane parallel to said base, said axis of movement and said guide pin (15) are collinear or said axis of movement diverges from said guide axis (15).

5. The injection device according to claim 1, characterized in that said guide element is configured such that the spine of said bird is placed in said exclusion zone, when at least the upper part of the back of the bird to be vaccinated is pressed against said base and that its neck is kept aligned or substantially aligned along this guide axis (15) in said injection zone.

6. The injection device according to claim 1, characterized in that, in line with the injecting position, the distance separating the free end of said injection needle from the exterior surface of said base (13) determines subcutaneous injection into the neck of said bird.

7. The injection device according to claim 1, characterized in that this injection device is configured such that its main body being placed on a flat horizontal surface, the bird is injected in the vertical or substantially vertical position.

8. The injection device according to claim 1, characterized in that said guide element (12) comprises a first part of a flared body and a second part of the body forming a narrowing, said first part of the body being arranged to guide the upper part of the body of the bird toward said narrowing.

9. The injection device according to claim 1, characterized in that said main body (11) comprises recesses (20) arranged laterally to this guide element in order to facilitate the insertion of the bird to be injected by the operator into this guide element.

10. The injection device according to claim 1, characterized in that each injection assembly comprising a linear actuator, said linear actuator is a hydraulic, pneumatic or electric actuator.

11. The injection device according to claim 1, characterized in that the needle is offset relative to the syringe(s).

12. The injection device according to claim 1, characterized in that it comprises two syringes and an injection needle.

13. The injection device according to claim 1, characterized in that the lower end of said guide element (12) is in communication with a slide (19) ensuring the discharging of the bird after the injection has been administered.

14. The injection device according to claim 1, characterized in that the corners of the parts of the device with which the bird and the operator are able to come into contact are rounded.

15. The injection device according to claim 1, characterized in that the upper end of said main body has an elbow to impose a particular inclination on the head of the bird, this elbow comprising one or more holding elements configured to keep the head of the bird to be vaccinated in position.
